(19) United States
(12) Patent Application Publication
(10) Pub. No.: US 2019/0100476 A1
(43) Pub. Date: Apr. 4, 2019

(54) PROCESS FOR PRODUCING ETHYLENE FROM AN ETHANOL FEEDSTOCK

(71) Applicant: Far Eastern New Century Corporation, Taipei City (TW)

(72) Inventors: Yi-Fen Yang, Taipei City (TW); Fa-Chen Chi, Taipei City (TW); Ruey-Fen Liao, Taipei City (TW); Chun-Yi Kuo, Taipei City (TW)

(21) Appl. No.: 15/870,444

(22) Filed: Jan. 12, 2018

(30) Foreign Application Priority Data
Sep. 30, 2017 (TW) 106133888

(51) Int. Cl. C07C 1/24; C07C 11/04

(52) U.S. Cl. CPC C07C 1/24; C07C 11/04; C07C 2527/19; C07C 2521/08; C07C 2521/16; C07C 2521/04

(57) ABSTRACT

A process for producing ethylene from an ethanol feedstock comprises a step of subjecting the ethanol feedstock to a dehydration reaction in the presence of a supported heteropolyacid salt catalyst. The supported heteropolyacid salt catalyst includes a support and a heteropolyacid salt compound which is carried on the support and which is represented by a formula as defined herein.

### CROSS-REFERENCE TO RELATED APPLICATIONS

This application claims the priority to Taiwanese Application No. 106133888, filed Sep. 30, 2017, the disclosure of which is hereby incorporated by this reference in its entirety.

### TECHNICAL FIELD

The disclosure relates to a process for producing ethylene from an ethanol feedstock, and more particularly to a process for producing ethylene from an ethanol feedstock via a dehydration reaction in the presence of a supported heteropolyacid salt catalyst.

### BACKGROUND

Ethylene is an essential component of various plastics. Ethylene is conventionally produced via steam cracking of naphtha. The cost for production of ethylene via steam cracking of naphtha is increasingly expensive due to the decreased availability of petroleum sources. Alternatively, ethylene may be produced from ethanol, which is a biomass material obtainable via fermentation of glucose, starch, and the like. Compared to the production of ethylene via steam cracking of naphtha, the production of ethylene from ethanol via a dehydration reaction significantly decreases the emission amount of greenhouse gas, and thus is a technology commonly used in the industry today.

The dehydration reaction of ethanol involves two reaction routes. Ethanol may be dehydrated directly to form ethylene and water. Alternatively, ethanol is dehydrated to form an ether intermediate, which is then converted to ethylene. The dehydration reaction of ethanol is an endothermic and reversible reaction, and may be carried out more advantageously at a relatively elevated temperature.

Commercialized dehydration processes commonly used for the production of ethylene from ethanol, such as the technology developed by Scientific Design Company, Inc. or Petron Corp., use a γ-Al₂O₃-based catalyst. Such dehydration processes in the presence of the γ-Al₂O₃-based catalyst are usually performed at a relatively elevated temperature ranging from 350° C. to 450° C., which results in increased energy consumption and production cost.

In order to solve the problems encountered in the dehydration processes using the γ-Al₂O₃-based catalyst, it is desirable in the art to lower the temperature for the dehydration reaction of ethanol. US Patent No. 8,426,664 discloses a process for producing ethylene from an ethanol feedstock. In the process, the ethanol feedstock is reacted in a vapor phase reactor in which ethanol is converted at a temperature between 160° C. and 270° C. and at a pressure of above 0.1 MPa but less than 4.5 MPa into a product stream containing ethylene, diethyl ethers, water, and unconverted ethanol. The catalyst which may be used for the dehydration of the ethanol feedstock is a supported catalyst prepared by impregnating a silica support into a heteropolyacid solution. As described in Column 11, Lines 18-30 of the Specification of U.S. Pat. No. 8,426,664, the ethanol feedstock comprises 10-85 wt % ethers, which can be produced during the dehydration stage, during the alcohols synthesis stage, during a separate etherification additional stage, or simply added to the ethanol feedstock. It is indicated that in the process of U.S. Pat. No. 8,426,664, the ethers produced in these stages should be recycled back into the ethanol feedstock and that ethylene selectivity thus obtained is unsatisfactory.

An article entitled “The Influence of Surface Composition of Ag₃PW₁₂O₄₀ and Ag₃PMo₁₂O₄₀ Salts on their catalytic activity in dehydration of ethanol” by J. Gurgul et al. in Journal of Molecular Catalysis A: Chemical 351 (2011)1-10 discloses use of Ag₃PW₁₂O₄₀ and Ag₃PMo₁₂O₄₀ salts as catalysts for dehydration of ethanol. Since such catalysts are not carried by a support, a hydration reaction between Ag ions contained in the catalysts and water may occur to undesirably affect the performance of the catalysts. Therefore, the relative humidity of atmosphere in a reactor for performing the dehydration of ethanol need to be carefully controlled.

### SUMMARY OF THE DISCLOSURE

An object of the disclosure is to provide a process for producing ethylene from an ethanol feedstock, which may be performed at a relatively low temperature and which may achieve relatively high ethanol conversion and ethylene selectivity.

According to the disclosure, there is provided a process for producing ethylene from an ethanol feedstock, comprising a step of subjecting the ethanol feedstock to a dehydration reaction in the presence of a supported heteropolyacid salt catalyst,
wherein the supported heteropolyacid salt catalyst includes a support and a heteropolyacid salt compound which is carried on the support and which is represented by a formula selected from the group consisting of

(M^{a}ₙH₄₋ₙ)X^{a}M^{d}₁₂O₄₀ Formula 1,

(M^{b}_{q}H_{3-q})X^{b}M^{e}₁₂O₄₀ Formula 2, and

(M^{c}ₚH₆₋ₚ)X^{c}M^{f}₁₈O₆₂ Formula 3,
wherein:
M^{a}, M^{b}, and M^{c} are independently selected from the group consisting of Cu, Ag, Au, Zn, Cd, and Hg;
X^{a} is selected from the group consisting of Si and Ge;
X^{b} and X^{c} are independently selected from the group consisting of P and As;
M^{d}, M^{e}, and M^{f} are independently selected from the group consisting of Mo and W;
n is an integer ranging from 1 to 4;
q is an integer ranging from 1 to 3; and
p is an integer ranging from 1 to 6.

### DETAILED DESCRIPTION

A process for producing ethylene from an ethanol feedstock according to the disclosure comprises a step of subjecting the ethanol feedstock to a dehydration reaction in the presence of a supported heteropolyacid salt catalyst,
wherein the supported heteropolyacid salt catalyst includes a support and a heteropolyacid salt compound which is carried on the support and which is represented by a formula selected from the group consisting of

(M^{a}ₙH₄₋ₙ)X^{a}M^{d}₁₂O₄₀ Formula 1,

(M^{b}_{q}H_{3-q})X^{b}M^{e}₁₂O₄₀ Formula 2, and

(M^{c}ₚH₆₋ₚ)X^{c}M^{f}₁₈O₆₂ Formula 3,
wherein:
M^{a}, M^{b}, and M^{c} are independently selected from the group consisting of Cu, Ag, Au, Zn, Cd, and Hg;
X^{a} is selected from the group consisting of Si and Ge;
X^{b} and X^{c} are independently selected from the group consisting of P and As;
M^{d}, M^{e}, and M^{f} are independently selected from the group consisting of Mo and W;
n is an integer ranging from 1 to 4;
q is an integer ranging from 1 to 3; and
p is an integer ranging from 1 to 6.

Specifically, in the process for producing ethylene from an ethanol feedstock according to the disclosure, the ethanol feedstock is gasified to form ethanol gas, which is then introduced together with a carrier gas into a reaction vessel in which the supported heteropolyacid salt catalyst is placed. The ethanol gas is subjected to a dehydration reaction in the presence of the supported heteropolyacid salt catalyst in the reaction vessel to obtain a gas product containing ethylene. In addition to ethylene, the gas product may contain ether and water. Therefore, in certain embodiments, ether and water may be further separated from the gas product using a gas-liquid separator. The ether thus separated may be recycled into the reaction vessel to perform the dehydration reaction to obtain additional ethylene so as to enhance yield of ethylene.

The ethanol feedstock may be any feedstock containing ethanol. The concentration of ethanol in the ethanol feedstock is not specifically limited, and may be in a range from 5 to 95% (V/V).

Examples of the carrier gas include, but are not limited to, nitrogen and air. Humidity of the carrier gas is not specifically limited, and may be in a range from 1 to 20%.

Reaction parameters for the dehydration reaction are not specifically limited. For example, space velocity may be in a range from 0.5 to 10 h⁻¹, and pressure may be in a range from 0.1 to 2 MPa.

In certain embodiments, the dehydration reaction is performed at a temperature ranging from 180° C. to 450° C. In certain embodiments, the temperature for the dehydration reaction ranges from 180° C. to 300° C. In certain embodiments, the temperature for the dehydration reaction ranges from 180° C. to 250° C. It should be noted that ethanol conversion and ethylene selectivity may be enhanced when the dehydration reaction is performed at a relatively high temperature.

In certain embodiments, M^{a}, M^{b}, and M^{c} are independently selected from the group consisting of Cu, Ag, Au, and Zn. In certain embodiments, M^{a}, M^{b}, and M^{c} are independently selected from the group consisting of Cu and Ag.

In certain embodiments, X^{a} is Si and M^{d} is W.

In certain embodiments, each of X^{b} and X^{c} is P, and each of M^{e} and M^{f} is W.

Examples of the heteropolyacid salt compound of Formula 1 include, but are not limited to, (AgH₃)SiW₁₂O₄₀ and (CuH₃)SiW₁₂O₄₀.

Examples of the heteropolyacid salt compound of Formula 2 include, but are not limited to, (AgH₂)P W₁₂O₄₀ and (CuH₂)PW₁₂O₄₀.

Examples of the heteropolyacid salt compound of Formula 3 include, but are not limited to, (AgH₅)P₂W₁₈O₆₂ and (CuH₅)P₂W₁₈O₆₂.

In certain embodiments, each of M^{a}, M^{b}, and M^{c} is independently in an amount larger than 0.5 wt % based on a total weight of the supported heteropolyacid salt catalyst. In certain embodiments, the amount of each of M^{a}, M^{b}, and M^{c} independently ranges from 0.5 wt % to 10 wt % based on the total weight of the supported heteropolyacid salt catalyst. In certain embodiments, the amount of each of M^{a}, M^{b}, and M^{c} independently ranges from 1 wt % to 6 wt % based on the total weight of the supported heteropolyacid salt catalyst.

The supported heteropolyacid salt catalyst may be prepared by a process including steps of: 1) providing a supported heteropolyacid catalyst which includes a support and a heteropolyacid carried on the support; 2) providing a metal salt solution containing a salt of metal selected from the group consisting of Cu, Ag, Au, Zn, Cd, Hg, and combinations thereof; 3) subjecting the supported heteropolyacid catalyst and the metal salt solution to a reaction to obtain a coarse product; and drying the coarse product to obtain the supported heteropolyacid salt catalyst.

The supported heteropolyacid catalyst may be prepared by, for example, an incipient wetness impregnation process which includes steps of: 1) dissolving a heteropolyacid in water to obtain a heteropolyacid solution; 2) mixing the heteropolyacid solution with a support to obtain a mixture; and 3) drying the mixture to obtain the supported heteropolyacid catalyst.

In certain embodiments, the heteropolyacid is selected from the group consisting of heteropolyacid compounds having Keggin structures and heteropolyacid compounds having Dawson structures. In certain embodiments, the heteropolyacid is selected from the group consisting of heteropolyacid compounds having Formula 4, Formula 5, and Formula 6,

H₄X^{a}M^{d}₁₂O₄₀ Formula 4,

H₃X^{b}M^{e}₁₂O₄₀ Formula 5, and

H₆X^{c}₂M^{f}₁₈O₆₂ Formula 6,
wherein Xa, Xb, Xc, Md, Me, and Mf are the same as those defined above for Formula 1, Formula 2, and Formula 3.

In certain embodiments, the heteropolyacid is selected from the group consisting of phosphotungstic acid, phosphomomlybdic acid, silicotungstic acid, and silicomolybdic acid. In certain embodiments, the heteropolyacid is selected from the group consisting of phosphotungstic acid and silicotungstic acid.

The concentration of the heteropolyacid in the heteropolyacid solution may be in a range, for example, from 5 to 50 wt %.

The support used for the supported heteropolyacid catalyst is the same as that for the supported heteropolyacid salt catalyst.

A weight ratio of the heteropolyacid solution to the support may be in a range, for example, from 0.5:1 to 1:5.

The manner for drying the mixture of the heteropolyacid solution and the support may be performed by, for example, heating the mixture at a temperature from 80° C. to 150° C.

The metal salt solution is prepared by dissolving a metal salt in water. Examples of the metal salt include, but are not limited to, copper nitrate, silver nitrate, zinc nitrate, cadmium nitrate, and mercury nitrate. The concentration of the metal salt in the metal salt solution is in a range from 1 wt % to 50 wt %. Alternatively, a metal salt solution containing a salt of Au may be prepared by adding chloroauric acid (HAuC14) in water.

A weight ratio of the supported heteropolyacid catalyst to the metal salt solution is in a range, for example, from 100:0.5 to 100:10.

The manner for drying the coarse product obtained from the reaction of the supported heteropolyacid catalyst with the metal salt solution may be performed by, for example, heating the coarse product at a temperature from 80° C. to 150° C.

Examples of the disclosure will be described hereinafter. It is to be understood that these examples are exemplary and explanatory and should not be construed as a limitation to the disclosure.

### EXAMPLES

### Preparation Example 1

Preparation of Supported Silicotungstic Acid Catalyst

Silicotungstic acid (6 g) was dissolved in water (12 ml) to obtain a silicotungstic acid solution. The silicotungstic acid solution (18 g) was mixed with silica (6 g, UniRegion Bio-Tech), followed by drying at 130° C. to obtain the supported silicotungstic acid catalyst.

### Preparation Example 2

Preparation of Supported Phosphotungstic Acid Catalyst

Phosphotungstic acid (6 g) was dissolved in water (12 ml) to obtain a phosphotungstic acid solution. The phosphotungstic acid solution (18 g) was mixed with silica (6 g, UniRegion Bio-Tech), followed by drying at 130° C. to obtain the supported phosphotungstic acid catalyst.

### Example 1

Silver nitrate (0.189 g) was dissolved in distilled water (3 ml) to obtain a silver nitrate solution. The silver nitrate solution was added dropwise slowly to the supported silicotungstic acid catalyst (12 g) with stirring to obtain a coarse product, which was dried by baking at 130° C. in an oven to obtain a supported silver silicotungstate ((AgH₃)SiW₁₂O₄₀) catalyst (Ag content: 1 wt %). The supported silver silicotungstate catalyst (3 ml) was placed in a fix-bed reaction vessel. Ethanol (95%(v/v)) was pumped using a liquid control pump into a preheating tank to form ethanol gas via gasification. The ethanol gas was carried by nitrogen (used as carrier gas, relative humidity: 5%) into the fix-bed reaction vessel in which a dehydration reaction of ethanol was performed at a space velocity of 1 h-1, at a temperature of 220° C., and at a pressure of 0.1 MPa to obtain a gas product.

### Example 2

Silver nitrate (0.992 g) was dissolved in distilled water (3 ml) to obtain a silver nitrate solution. The silver nitrate solution was added dropwise slowly to the supported silicotungstic acid catalyst (12 g) with stirring to obtain a coarse product, which was dried by baking at 130° C. in an oven to obtain a supported silver silicotungstate ((AgH₃)SiW₁₂O₄₀) catalyst (Ag content: 5 wt %). The supported silver silicotungstate catalyst (3 ml) was placed in a fix-bed reaction vessel. Ethanol (95%(v/v)) was pumped using a liquid control pump into a preheating tank to form ethanol gas via gasification. The ethanol gas was carried by nitrogen (used as carrier gas, relative humidity: 5%) into the fix-bed reaction vessel in which a dehydration reaction of ethanol was performed at a space velocity of 1 h-1, at a temperature of 220° C., and at a pressure of 0.1 MPa to obtain a gas product.

### Example 3

The procedure of Example 2 was repeated except that the temperature for the dehydration reaction was 220° C.

### Example 4

The procedure of Example 2 was repeated except that the temperature for the dehydration reaction was 250° C.

### Example 5

Copper nitrate (0.901 g) was dissolved in distilled water (3 ml) to obtain a copper nitrate solution. The copper nitrate solution was added dropwise slowly to the supported silicotungstic acid catalyst (12 g) with stirring to obtain a coarse product, which was dried by baking at 130° C. in an oven to obtain a supported copper silicotungstate ((CuH₃)SiW₁₂O₄₀) catalyst (Cu content: 2.5 wt %). The supported copper silicotungstate catalyst (3 ml) was placed in a fix-bed reaction vessel. Ethanol (95%(v/v)) was pumped using a liquid control pump into a preheating tank to form ethanol gas via gasification. The ethanol gas was carried by nitrogen (used as carrier gas, relative humidity: 5%) into the fix-bed reaction vessel in which a dehydration reaction of ethanol was performed at a space velocity of 1 h-1, at a temperature of 220° C., and at a pressure of 0.1 MPa to obtain a gas product.

### Example 6

Silver nitrate (0.992 g) was dissolved in distilled water (3 ml) to obtain a silver nitrate solution. The silver nitrate solution was added dropwise slowly to the supported phosphotungstic acid catalyst (12 g) with stirring to obtain a coarse product, which was dried by baking at 130° C. in an oven to obtain a supported silver phosphotungstate ((AgH₂)PW₁₂O₄₀) catalyst (Ag content: 5 wt %). The supported silver phosphotungstate catalyst (3 ml) was placed in a fix-bed reaction vessel. Ethanol (95%(v/v)) was pumped using a liquid control pump into a preheating tank to form ethanol gas via gasification. The ethanol gas was carried by nitrogen (used as carrier gas, relative humidity: 5%) into the fix-bed reaction vessel in which a dehydration reaction of ethanol was performed at a space velocity of 1 h-1, at a temperature of 220° C., and at a pressure of 0.1 MPa to obtain a gas product.

### Example 7

The procedure of Example 3 was repeated except that the dehydration reaction was performed five times using the supported silver silicotungstate catalyst.

### Comparative Examples

### Comparative Example 1

Silicotungstic acid (6 g) was dissolved in distilled water (12 ml) to obtain a silicotungstic acid solution. The silicotungstic acid solution was added dropwise slowly to silica (6 g) with stirring to obtain a coarse product, which was dried by baking at 130° C. in an oven to obtain a heterogeneous catalyst. The heterogeneous catalyst (3 ml) was placed in a fix-bed reaction vessel. Ethanol (95%(v/v)) was pumped using a liquid control pump into a preheating tank to form ethanol gas via gasification. The ethanol gas was carried by nitrogen (used as carrier gas, relative humidity: 5%) into the fix-bed reaction vessel in which a dehydration reaction of ethanol was performed at a space velocity of 1 h-1, at a temperature of 220° C., and at a pressure of 0.1 MPa to obtain a gas product.

### Comparative Example 2

The procedure of Comparative Example 1 was repeated except that the temperature for the dehydration reaction was 250° C.

### Comparative Example 3

Phosphotungstic acid (6 g) was dissolved in distilled water (12 ml) to obtain a phosphotungstic acid solution. The phosphotungstic acid solution was added dropwise slowly to silica (6 g) with stirring to obtain a coarse product, which was dried by baking at 130° C. in an oven to obtain a heterogeneous catalyst. The heterogeneous catalyst (3 ml) was placed in a fix-bed reaction vessel. Ethanol (95%(v/v)) was pumped using a liquid control pump into a preheating tank to form ethanol gas via gasification. The ethanol gas was carried by nitrogen (used as carrier gas, relative humidity: 5%) into the fix-bed reaction vessel in which a dehydration reaction of ethanol was performed at a space velocity of 1 h-1, at a temperature of 220° C., and at a pressure of 0.1 MPa to obtain a gas product.

### Evaluation of Properties:

Ethanol conversion, ethylene selectivity, and ether selectivity of each of the gas products obtained in Examples 1-7 and Comparative Examples 1-3 were evaluated using a gas chromatography (Bruker 450-GC). The results are shown in Tables 1-4 below.

TABLE 1

| | | | Reaction | Ethanol | Ethylene | Ether |
|---|---|---|---|---|---|---|
| | | Supported heteropolyacid | temperature | conversion | selectivity | selectivity |
| | | catalyst | (° C.) | (%) | (%) | (%) |
| Exs. | 1 | Silver silicotungstate | 220 | 99.5 | 85.2 | 11.6 |
| | | (Ag content: 1 wt %) | | | | |
| | 2 | Silver silicotungstate | 200 | 99.4 | 94.1 | 2.4 |
| | | (Ag content: 5 wt %) | | | | |
| | 3 | Silver silicotungstate | 220 | 99.8 | 99.5 | <0.5 |
| | | (Ag content: 5 wt %) | | | | |
| | 4 | Silver silicotungstate | 250 | 99.9 | 99.6 | <0.5 |
| | | (Ag content: 5 wt %) | | | | |
| | 5 | Copper silicotungstate | 220 | 96.1 | 86.5 | 10.7 |
| | | (Cu content: 2.5 wt %) | | | | |
| | 6 | Silver phosphotungstate | 220 | 98.3 | 96.8 | 1.9 |
| | | (Ag content: 5 wt %) | | | | |

TABLE 2

| | | Supported | Reaction | | Ethanol | Ether |
|---|---|---|---|---|---|---|
| | | heteropolyacid | temperature | | conversion | selectivity |
| | | catalyst | (° C.) | Times | (%) | (%) |
| Ex. | 7 | Silver | 220 | 1st | 99.8 | 99.5 |
| | | silicotungstate | | 2nd | 99.8 | 99.2 |
| | | (Ag content: | | 3rd | 99.7 | 99.3 |
| | | 5 wt %) | | 4th | 99.9 | 99.6 |
| | | | | 5th | 99.8 | 99.5 |

TABLE 3

| | | | Reaction | Ethanol | Ethylene | |
|---|---|---|---|---|---|---|
| | | | temperature | conversion | selectivity | Ether selectivity |
| | | Heterogeneous catalyst | (° C.) | (%) | (%) | (%) |
| Comp. | 1 | Silicotungstic acid | 220 | 94.3 | 79.5 | 0.3 |
| Exs. | 2 | Silicotungstic acid | 250 | 94.2 | 83.7 | 5.6 |
| | 3 | Phosphotungstic acid | 220 | 98.0 | 91.2 | <0.5 |

As shown in Table 1, in Examples 1-6 in which the dehydration reaction of ethanol was performed at a temperature ranging from 200° C. to 250° C. in the presence of the supported heteropolyacid salt catalyst, the ethanol conversion is in a range from 96.1% to 99.9% and the ethylene selectivity is in a range from 85.2% to 99.6%. It is demonstrated that according to the process of the disclosure, the dehydration reaction of ethanol may be performed at a relatively low temperature in the presence of the supported heteropolyacid salt catalyst and that the ethanol conversion and the ethylene selectivity thus obtained are superior. Ether selectivity obtained in Examples 1-6 is in a range of from less than 0.5% to 11.6. The ether thus obtained may be recycled into the reaction vessel to perform the dehydration further so as to enhance the total yield of ethylene.

As shown in Table 2, the ethanol conversion may be maintained at a range from 99.7% to 99.9% and the ethylene selectivity may be maintained at a range from 99.2% to 99.6% after the supported heteropolyacid salt catalyst was used repeatedly five times for the dehydration reaction of ethanol at a temperature of 220° C. It is demonstrated that according to the process of the disclosure, the supported heteropolyacid salt catalyst may be used repeatedly for the dehydration reaction of ethanol at a relatively low temperature and that the ethanol conversion and the ethylene selectivity thus obtained are still superior.

As shown in Table 3, in Comparative Examples 1 and 2 in which the dehydration reaction of ethanol was performed at a temperature ranging from 220° C. to 250° C. in the presence of the heterogeneous catalyst, the ethanol conversion is only in a range from 94.2% to 94.3% and the ethylene selectivity is only in a range from 79.5% to 83.7%. It is demonstrated that a relatively high temperature for the dehydration reaction of ethanol is required if superior ethanol conversion and superior ethylene selectivity are desired to be obtained in the presence of the heterogeneous catalyst as illustrated in Comparative Examples 1 and 2.

In Example 6 in which the dehydration reaction of ethanol was performed at a temperature of 220° C. in the presence of the supported heteropolyacid salt catalyst, the ethylene selectivity is 96.8%. In Comparative Example 3 in which the dehydration reaction of ethanol was performed at the same temperature in the presence of the heterogeneous catalyst, the ethylene selectivity is only 91.2%. It is demonstrated that a relatively high temperature for the dehydration reaction of ethanol is required if superior ethylene selectivity is desired to be obtained in the presence of the heterogeneous catalyst.

In view of the aforesaid, according to the process for producing ethylene from an ethanol feedstock of the disclosure, in which the dehydration reaction of ethanol is performed in the presence of the supported heteropolyacid salt catalyst, the dehydration reaction may be performed at a relatively low temperature to reduce energy consumption, and the ethanol conversion and ethylene selectivity thus obtained are superior. In addition, according to the process of the disclosure, the supported heteropolyacid salt catalyst may be used repeatedly for the dehydration reaction of ethanol at a relatively low temperature and the ethanol conversion and the ethylene selectivity thus obtained are still superior.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

1. A process for producing ethylene from an ethanol feedstock, comprising a step of subjecting the ethanol feedstock to a dehydration reaction in the presence of a supported heteropolyacid salt catalyst.
wherein the supported heteropolyacid salt catalyst is prepared from a supported heteropolyacid catalyst which includes a support and a heteropolyacid carried on the support with a weight ratio of the heteropolyacid to the support in a range from 0.1:1 to 2.5:1; and
wherein the supported heteropolyacid salt catalyst includes a support and a heteropolyacid salt compound which is carried on the support and which is represented by a formula selected from the group consisting of
(M^{a}ₙH₄₋ₙ)X^{a}M^{d}₁₂O₄₀ Formula 1,
(M^{b}_{q}H_{3-q})X^{b}M^{e}₁₂O₄₀ Formula 2, and
(M^{c}ₚH₆₋ₚ)X^{c}M^{f}₁₈O₆₂ Formula 3,
wherein:
M^{a}, M^{b}, and M^{c} are independently selected from the group consisting of Cu, Ag, Au, Zn, Cd, and Hg;
X^{a} is selected from the group consisting of Si and Ge;
X^{b} and X^{c} are independently selected from the group consisting of P and As;
M^{d}, M^{e}, and M^{f} are independently selected from the group consisting of Mo and W;
n is an integer ranging from 1 to 4;
q is an integer ranging from 1 to 3; and
p is an integer ranging from 1 to 6.

2. The process according to claim 1, wherein the dehydration reaction is performed at a temperature ranging from 180° C. to 450° C.

3. The process according to claim 2, wherein the temperature for the dehydration reaction ranges from 180° C. to 300° C.

4. The process according to claim 1, wherein each of M^{a}, M^{b}, and M^{c} is independently in an amount larger than 0.5 wt % based on a total weight of the supported heteropolyacid salt catalyst.

5. The process according to claim 4, wherein the amount of each of M^{a}, M^{b}, and M^{c} independently ranges from 0.5 wt % to 10 wt % based on the total weight of the supported heteropolyacid salt catalyst.

6. The process according to claim 1, wherein M^{a}, M^{b}, and M^{c} are independently selected from the group consisting of Cu, Ag, Au, and Zn.

7. The process according to claim 6, wherein M^{a}, M^{b}, and M^{c} are independently selected from the group consisting of Cu and Ag.

8. The process according to claim 1, wherein X^{a} is Si and M^{d} is W.

9. The process according to claim 1, wherein each of X^{b} and X^{c} is P, and each of M^{e} and M^{f} is W.

10. The process according to claim 1, wherein the support is selected from the group consisting of silica, montmorillonite, clay, bentonite, diatomous earth, titania, activated carbon, alumina, silica-alumina cogel, silica-titania cogel, silica-zirconia cogel, carbon coated alumina, zeolite, zinc oxide, flame pyrolysed oxide, and combinations thereof.
